(19) United States
(12) Patent Application Publication
(10) Pub. No.: US 2009/0017505 A1
(43) Pub. Date: Jan. 15, 2009

(54) Process and device for mixing microdroplets

(75) Inventors: Ulrich Sieben, Vorstetten (DE); Holger Klapproth, Freiburg (DE)

Correspondence Address: THE WEBB LAW FIRM, P.C., 700 KOPPERS BUILDING, 436 SEVENTH AVENUE, PITTSBURGH, PA, 15219, US

(73) Assignee: Micronas Holding GmbH, Freiburg i.Br. (DE)

(21) Appl. No.: 11/805,242

(22) Filed: May 22, 2007

(30) Foreign Application Priority Data
May 22, 2006 (EP) 06010516

(51) Int. Cl. C12P 19/34; C12M 1/40; C12M 1/34

(52) U.S. Cl. 435/91.2; 435/299.1; 435/287.1

(57) ABSTRACT

In a process for mixing microdroplets (2a, 2b, 2c), at least two carriers (3a, 3b) are provided whose surfaces (4a, 4b) are each structured in such a way that at least one hydrophilic surface domain (5a, 5b) is delimited by at least one hydrophobic surface domain (6a, 6b). A first microdroplet (2a, 2b, 2c) is disposed on a hydrophilic surface domain (5a) of a first carrier (3a), and a second microdroplet (2a, 2b, 3c) is disposed on a hydrophilic surface domain (5b) of a second carrier (3b). The carriers (3a, 3b), with their first and second hydrophilic surfaces (4a, 4b) facing each other, are positioned adjacent to each other and sufficiently close to each other by being moved toward each other that the microdroplets (2a, 2b, 2c) come into contact with each other.

The invention relates to a process and a device for mixing microdroplets.

It is known from actual practice that quantities of liquids in the range of less than 1 μL can be dispensed with so-called nanopipettes. In order to mix microdroplets, a supply of a first liquid is maintained in a reservoir in a first nanopipette. A nozzle of the nanopipette that is connected to the reservoir by means of a channel is positioned on a substrate in order to eject the microdroplet of the liquid onto the substrate with the aid of an actuator. A second nanopipette is then positioned on the substrate in order to apply a second microdroplet of a second liquid to the substrate in a similar manner, in order, for example, to initiate a chemical reaction between the liquids. As soon as the microdroplets come into contact with each other, they mix together. Because of the small size of the microdroplets, however, it is difficult to place the second microdroplet precisely at the same position on the substrate as the first microdroplet. This is particularly difficult with liquids that have a low viscosity, since in the case of these liquids the microdroplets can easily break apart upon emerging from the nozzle. If a plurality of microdroplets is to be applied to the substrate one after another, there is also a risk that the microdroplets will evaporate before they come into contact with each other. Moreover, the dispensing of the liquids is dependent on their surface energy. Thus, for example, liquids that have a high surface energy produce larger microdroplets than the liquids that have a low surface energy.

The object of the invention accordingly is to provide a process and a device that permit least two microdroplets to be mixed together in a simple way.

This object is accomplished with respect to the process thusly: At least two carriers are provided whose surfaces are each structured in such a way that at least one hydrophilic surface domain is delimited by at least one hydrophobic surface domain; a first microdroplet is disposed on a hydrophilic surface domain of a first carrier and a second microdroplet is disposed on a hydrophilic surface domain of a second carrier; and the carriers, with their first and second surfaces facing each other, are positioned adjacent to each other and sufficiently close to each other that the microdroplets come into contact with each other.

In a preferred manner a self-alignment of the liquids on the hydrophilic domains is accomplished for aqueous liquids by structuring the carrier surfaces into hydrophilic and hydrophobic domains. In this way, the microdroplets can be placed on the carriers with a high degree of positioning accuracy and precision. By positioning the carriers relative to each other in the correct orientation, the microdroplets are brought into contact with each other and are mixed together.

In a preferred embodiment the surface of a first carrier is provided with a preferably matrix-like surface structure that possesses a plurality of hydrophilic surface domains that are separated from each other by at least one hydrophobic surface domain, the surface of a second carrier is provided with a surface structure that coincides with the surface structure of the first carrier, one microdroplet is applied to each of the individual hydrophilic surface domains, and the carriers, with their surface structures facing each other, are positioned adjacent to each other by being moved toward each other in such a way that the microdroplets of corresponding hydrophilic surface domains each come into contact with each other. In this way, a plurality of microdroplets, which are oriented in pairs relative to each other, can be mixed together simultaneously when the carriers are positioned adjacent to each other.

With respect to the process, the object recited above is accomplished as follows: At least two carriers are provided; the surface of a first carrier is structured in such a way that hydrophilic surface domains that are adjacent to each other in close proximity to each other are separated from each other by at least one hydrophobic surface domain; the hydrophilic surface domains are each brought into contact with one microdroplet; a second carrier is positioned relative to the hydrophilic surface domains in such a way that the microdroplets come into contact with the second carrier and with each other.

When this occurs, the hydrophobic surface domain of the first carrier that is located between the hydrophilic surface domains that are adjacent to each other is spanned by the second carrier in such a way that the microdroplets come into contact with each other and mix together, for example because the microdroplets are laterally displaced when the carriers are positioned adjacent to each other and/or because they diffuse into the other corresponding microdroplet. The surface of the second carrier is preferably hydrophilic. The process can be performed in a particularly simple and cost-effective manner, since only one of the two carriers needs to be structured.

With respect to the process, the object recited above is also accomplished as follows: At least two carriers are provided, the surface of a first carrier is structured in such a way that first hydrophilic surface domains that are adjacent to each other in close vicinity to each other are separated from each other by at least one first hydrophobic surface domain; the surface of a second carrier is structured in such a way that at least one second hydrophilic surface domain is delimited by at least one second hydrophobic surface domain; the first hydrophilic surface domains and at least one second hydrophilic surface domain are each brought into contact with a microdroplet; and the carriers, with their first hydrophilic surface domains and at least one second hydrophilic surface domain facing each another, are positioned adjacent to each other and sufficiently close to each other by being moved toward each other, that the second hydrophilic surface domain overlaps an area of the first hydrophobic surface domain located between the first hydrophilic surface domains and that at least three microdroplets come into contact with each other.

Hence, in this solution to the problem, at least three microdroplets that are disposed on the carrier surfaces are mixed together with each other virtually simultaneously and in a simple manner.

It is advantageous if at least one of the carriers used for applying the microdroplet(s) to the hydrophilic surface domain(s) is immersed into a liquid and then preferably is drawn out of the liquid at a rate in the range of 0.1 to 10 mm/second. The hydrophilic surface domain, of which at least one is present, can thereby be loaded in a simple manner with the microdroplet. When the carrier is drawn out of the liquid, the liquid beads off of the hydrophobic surface domains, while it continues to adhere to the hydrophilic surface domains in the form of the microdroplet.

In a preferred embodiment of the invention, a first microdroplet contains an enzyme and a second microdroplet contains at least one DNA molecule, primer, and nucleoside triphosphate in a concentration that is sufficient for performing a polymerase chain reaction. A polymerase chain reaction may be initiated in a simple manner by bringing the microdroplets into contact with each other in order to amplify the DNA molecule. The process even permits a large number of polymerase chain reactions to be initiated simultaneously, in which case the individual reactions start as soon as the microdroplets are brought into contact with each other. In contrast to conventional methods, a so-called hot-start, in which the enzyme is inactivated by a thermolabile group upon the application of heat, is no longer necessary.

In another preferred embodiment of the invention, a first microdroplet contains hydrogen peroxide and the second microdroplet contains Luminol. The process may be used for the optical detection of receptor-ligand complexes that are directly or indirectly marked with an enzyme so that, when the complexes are present, the Luminol decomposes upon contact with the hydrogen peroxide emitting chemoluminescent radiation. The process may be used in particular with ELISA or sandwich ELISA processes.

It is advantageous for least one carrier to be provided as a metal oxide or semi-metal oxide substrate and for the substrate to be coated with a polymer having at least one reactive group at the sites at which the hydrophilic surface domains are to be located. The substrate may then be structured with great precision using methods of semiconductor manufacturing that are known per se. The reactive group may, for example, have an OH, SH, and/or NH2 group. The polymer may be a gel and in particular may contain a polysaccharide and/or poly(2-hydroxyethyl) methacrylate (pHEMA).

The object recited above is accomplished with respect to the device of the type referred to above as follows: The device has at least two carriers whose surfaces are each structured in such a way that at least one hydrophilic surface domain is delimited by at least one hydrophobic surface domain; the device has a positioning means by which the carriers, with their structured surfaces facing each other, may be positioned adjacent to each other and sufficiently close to each other that microdroplets that can be applied to the hydrophilic surface domains come into contact with each other.

By structuring the carrier surfaces into hydrophilic and hydrophobic domains, self-alignment of the microdroplets on the hydrophilic domains becomes possible when the carrier surface comes into contact with an aqueous liquid. With the aid of the positioning means, the hydrophilic surface domains of carriers can then be positioned adjacent to one another in a simple manner in such a way that the microdroplets come into contact with each other and mix together.

The object recited above is also accomplished with respect to the device stated above as follows: The device has at least two carriers; the surface of a first carrier is structured in such a way that hydrophilic surface domains that are adjacent to each other in close vicinity to each other are separated from each other by at least one hydrophobic surface domain; and the device has a positioning means, by which means the carriers can be positioned adjacent to each other and in close vicinity to each other in such a way that microdroplets that can be placed on the hydrophilic surface domains of the first carrier come into contact with the second carrier and with each other.

A self-alignment of the microdroplets is also made possible with this device through the structuring of the carrier surfaces into hydrophilic and hydrophobic domains. Since only one of the two carriers has to have surface structuring, the device can be manufactured economically.

With respect to the device of the type referred to above, the object recited above is also accomplished as follows: The device has at least two carriers, the surface of a first carrier is structured in such a way that first hydrophilic surface domains that are adjacent to each other in close vicinity to each other are separated from each other by at least one first hydrophobic surface domain; the surface of a second carrier is structured in such a way that at least one second hydrophilic surface domain is delimited by at least one second hydrophobic surface domain; the device has a positioning means, by which means the carriers, with their structured surfaces facing each other, may be positioned adjacent to each other and sufficiently close to each other that the second hydrophilic surface domain overlaps a first hydrophobic surface domain that is located between the first hydrophilic surface domains and that microdroplets that can be placed on the first hydrophilic surface domains come into contact with the microdroplets that can be placed on the second hydrophilic surface domains.

By means of the device it is therefore possible, in an easy manner, to bring three microdroplets into contact with each other virtually simultaneously and to mix them together.

It is advantageous for the device to have at least three of the carriers and for these carriers to be able to be positioned adjacent to each other by means of the positioning means, either as desired or alternatingly. In this way it is possible, in particular, to mix together a plurality of microdroplets one after another, for example to first mix together two microdroplets A and B to form microdroplet AB, and to then mix this with microdroplet C to form microdroplet ABC.

It is advantageous for at least one carrier to have a metal oxide or semi-metal oxide substrate that is coated on the hydrophilic surface domains with a least one polymer having a reactive group. The substrate may be mass-produced with a high degree of precision using the methods employed in manufacturing semiconductors.

In a preferred embodiment of the invention, the positioning means has centering elements, in particular inclined centering surfaces, that work together with each other on the carriers that are to be positioned adjacent to one another. The carriers may be positioned in a simple manner relative to one another with their surface structuring in a specified position. A projection may be provided on the one carrier and a matching recess may be provided on the other carrier to form a centering element. The centering elements may also be optical markings such as crosshairs that are brought into alignment when the carriers are positioned adjacent to one another.

It is advantageous for at least one carrier to preferably have a moisture and/or conductivity sensor at one hydrophilic surface domain. The sensor may be used in a simple manner to check whether the microdroplets have come into contact with each other, for example when the liquids of the microdroplets have different electrical conductivities.

In a preferred embodiment of the invention, at least one carrier has a cooling or heating element, in particular a Peltier element. The device may be used to perform a polymerase chain reaction (PCR).

Typical embodiments of the invention are described in greater detail below as examples. They show:

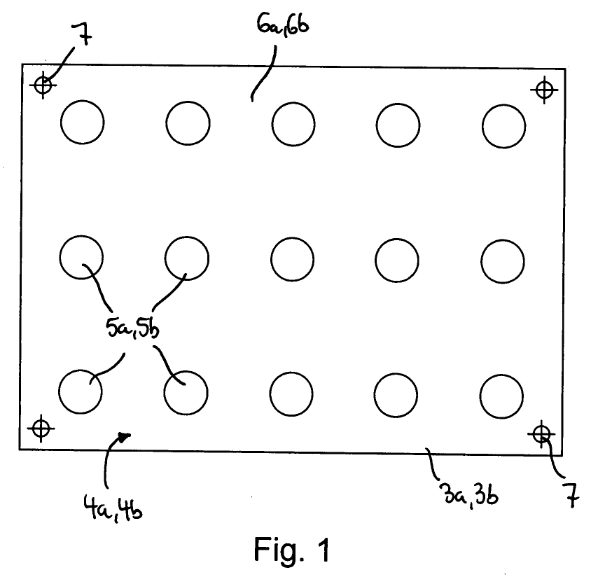

FIG. 1—a top view of a first carrier of a first example of the embodiment of a device for mixing microdroplets,

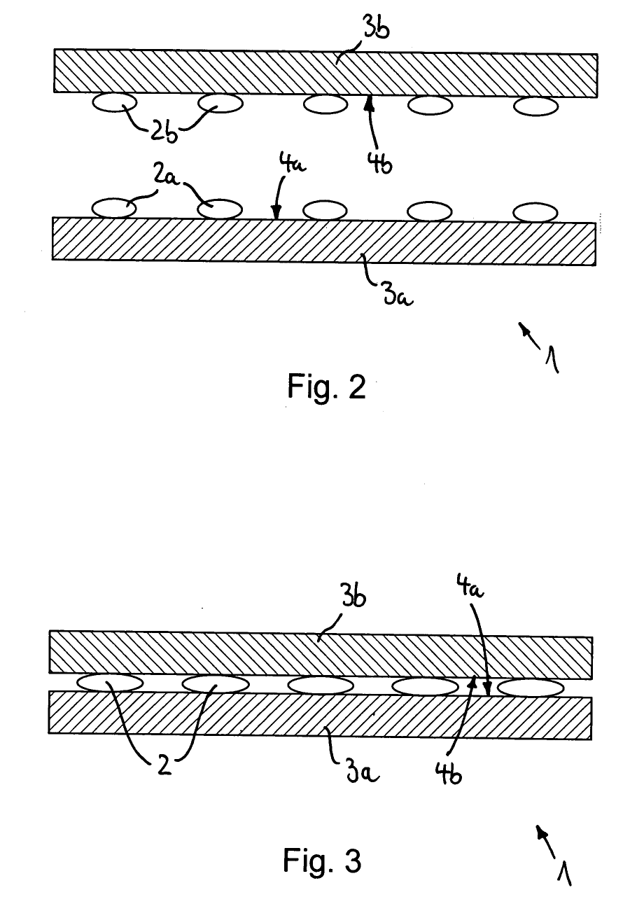

FIG. 2—a cross-sectional view through the microdroplet-coated carrier of the first example of an embodiment of the device, in which the carriers are located in the initial position,

FIG. 3—a diagram similar to that in FIG. 2, in which, however, the carriers have been moved from the initial position toward one another,

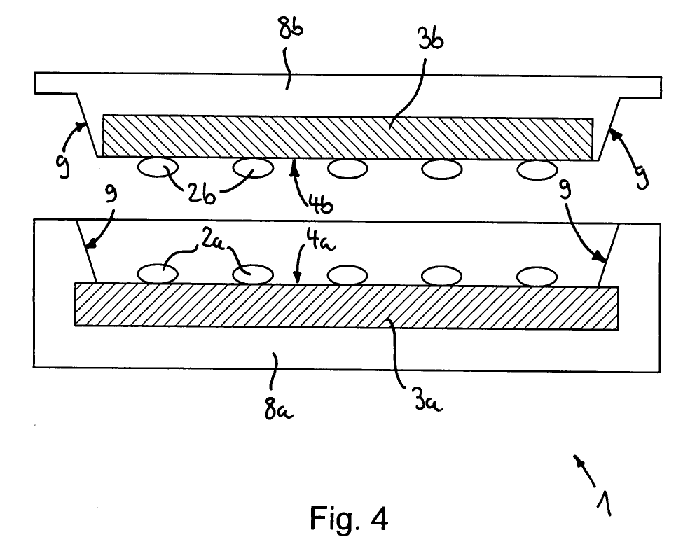

FIG. 4—a cross-sectional view through the carriers of a second example of an embodiment of the device,

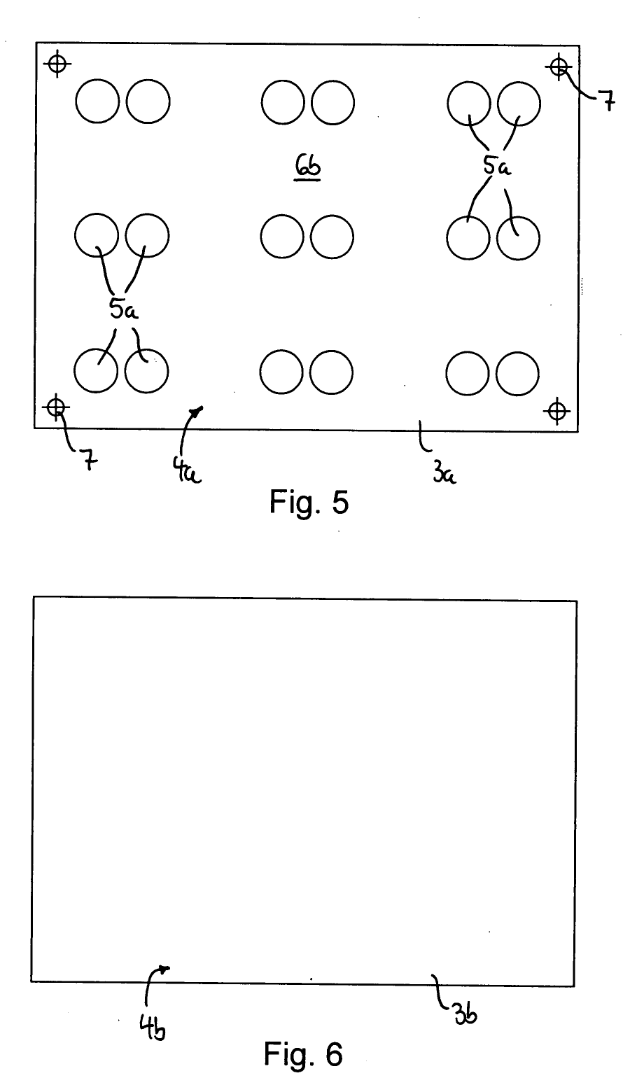

FIG. 5—a top view of a first carrier of a third example of an embodiment of the device,

FIG. 6—a top view of a second carrier of the third example of an embodiment of the device,

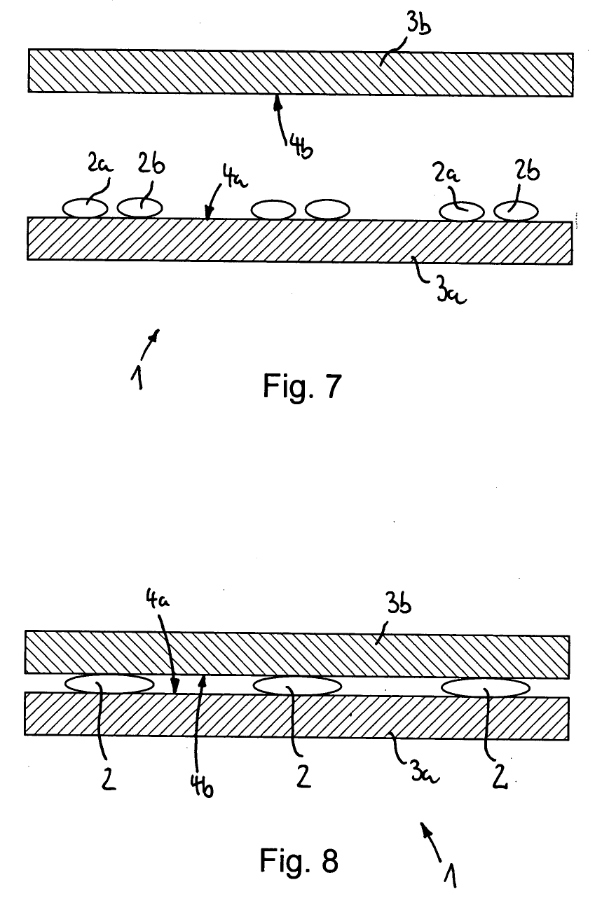

FIG. 7—a cross-sectional view through the microdroplet-coated carrier of the third example of an embodiment of the device, in which the carriers are located in an initial position,

FIG. 8—a diagram similar to that in FIG. 7, in which however the carriers have been moved from the initial position toward one another,

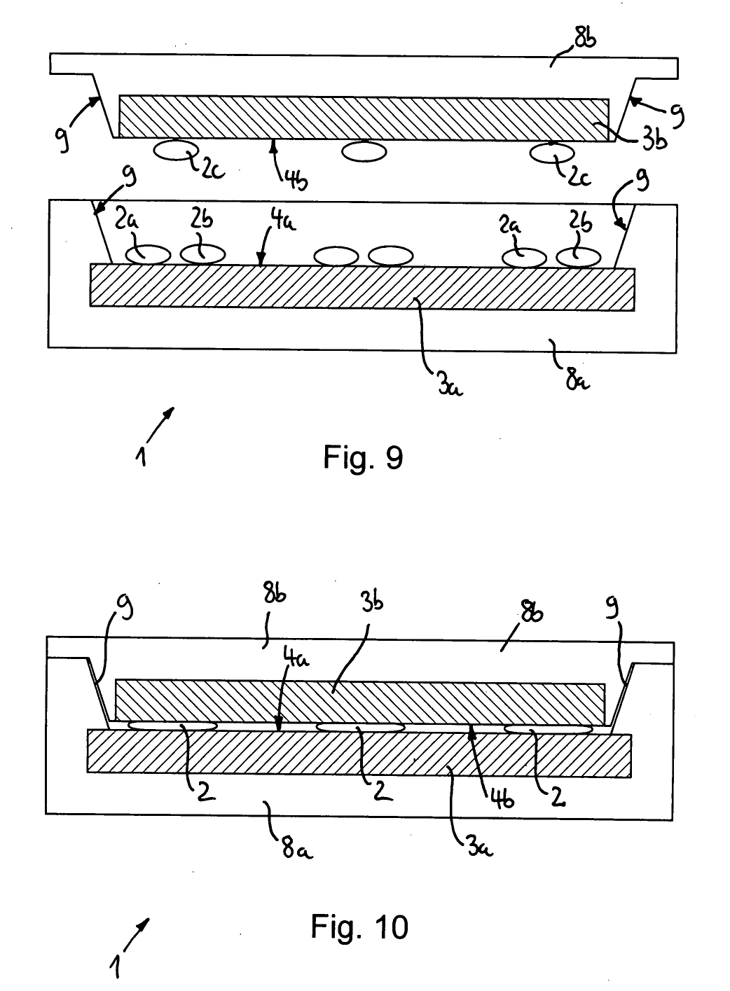

FIG. 9—a cross-sectional view through the microdroplet-coated carrier of a fourth example of embodiment of the device, in which the carriers are located in the initial position, and

FIG. 10—a diagram similar to that in FIG. 9, in which, however, the carriers have been moved from the initial position toward one another.

A device 1 shown in FIGS. 1 to 3 for mixing microdroplets 2a, 2b has two approximately plate-shaped carriers 3a, 3b, who surfaces 4a, 4b are structured in such a way that a plurality of hydrophilic surface domains 5a, 5b are laterally separated from each other by a hydrophobic surface domain 6a, 6b that delimits them. The hydrophilic surface domains 5a, 5b are arranged in the shape of matrices in a plurality of rows and columns. The matrices of the two carriers 3a, 3b are designed in such a way that the hydrophobic surface domains 6a, 6b of a first carrier 3a can be made to overlap those of a second carrier 3b if the carriers 3a, 3b are positioned adjacent to each other with their hydrophilic surface domains 5a, 5b facing one another.

In FIG. 1 one can see that the carrier has optical position marks that are embodied as crosshairs and that are disposed in a specified position relative to the hydrophilic surface domains 5a, 5b.

The carriers 3a, 3b each consist of a semiconductor material, such as silicon, that has on its surface a fluoropolymer layer, which is not shown in the drawing, that forms the hydrophobic surface domain 6a, 6b. A polymer hydrogel, which may have reactive groups, is applied to the fluoropolymer layer in each of the hydrophilic surface domains 5a, 5b.

First microdroplets 2a are applied to the hydrophilic surface domains 5a of the first carrier 3a. Carrier 3a, for example, can be immersed in a liquid and then withdrawn from this liquid at a speed that is selected so that the liquid continues to adhere only to the hydrophilic surface domains 5a. The microdroplets 2a may be applied, however, in any other desired way to the hydrophilic surface domains 5a, for example with the aid of a needle, a pipette, or by printing, in particular by means of a jet printer. Here the various surface domains 5a, 6a cause the microdroplets 2a to align with each other of their own accord so that they are only disposed on the hydrophilic surface domains 5a.

In a corresponding manner, second microdroplets 2b are applied to the hydrophilic surface domains 5a, 5b of the second carrier 3b. The carriers 3a, 3b, together with their planes of extension are positioned parallel to each other in such a way that the hydrophilic surface domains 5a of the first carrier 3a are symmetrically opposite to the hydrophilic surface domains 5b of the second carrier 3b. To align the carriers 3a, 3b in the correct position, the position marks 7 of the one carrier 3a are made to coincide with the position marks 7 of the other carrier 3b.

As can be seen in FIG. 2, carriers 3a, 3b initially are far enough apart that the microdroplets 2a, 2b do not touch each other. In the arrangement shown in FIG. 2, the microdroplets 2a are located on the top of the first carrier 3a, and the microdroplets 2b are located on the underside of the second carrier 3b. These latter droplets adhere to the hydrophilic surface domains 5b despite the force of gravity. Of course, it is also possible for the plate arrangement formed by the carriers 3a, 3b to be positioned in a different orientation in space, for example rotated by 90° about an axis that is normal to the plane of the drawing in FIGS. 2 and 3.

In a further process step the carriers 3a, 3b are moved toward each other by means of a positioning means, which is not shown in the drawing, a robot for example, normal to their planes of extension until the microdroplets 2a located on the surface of the first carrier 3a each contact a corresponding microdroplet 2b on the second carrier 3b, and mix with it, for example to initiate a chemical reaction between the various liquids in the microdroplets 2a, 2b, and/or substances dissolved therein. In FIG. 3 it can be seen that, after the microdroplets 2a, 2b are mixed together to form a new microdroplet 2, the carriers 3a, 3b are spaced apart from each other by means of a narrow intermediate space and that the microdroplets 2 are spaced apart from each other by means of the hydrophobic surface domains 6a, 6b. Microdroplets 2a, 2b that are disposed on the same carrier 3a, 3b are therefore not mixed together.

In the embodiment shown in FIG. 4, the positioning means has a first housing part 8a that is connected to the first carrier 3a and a second housing part 8b that is connected to the second carrier 3b. The first housing part 8a has a receiving recess, and the second housing part 8b has a matching projection. Inclined surfaces 9 are disposed on the housing parts 8a, 8b in such a way that, when the second housing part is inserted into the first housing part 8a, these inclined surfaces worked together to center the housing parts 8a, 8b in a specified position relative to each other. The housing parts 8a, 8b are preferably made of an inert plastic that is injected molded onto the carrier parts 3a, 3b in some areas.

In the example of the embodiment shown in FIGS. 5 to 8, hydrophilic surface domains 5a, which are separated from each other by a hydrophobic surface domain 6a, are only provided on the surface of the first carrier part 3a. The hydrophilic surface domains 5a are arranged in the shape of matrices in a plurality of rows and columns. FIG. 5 clearly shows that two surface domains 5a are each arranged in pairs relative to each other and that they have a smaller distance between them than they have relative to the other hydrophilic surface domains 5a. The surface of the second carrier part 3b, which serves as a male die, is completely hydrophilic.

A first microdroplet 2a is used up [typo in German (aufgebraucht) should probably read “aufgebracht” (applied)] on one surface domain 5a of the surface domains 5a that are arranged in pairs relative to each other, and the second microdroplet 2b is used up [sic: applied] on the other surface domain 5b. The application of the microdroplets 2a, 2b can be accomplished, for example, by means of printing.

As can be seen in FIG. 7, the carriers 3a, 3b and their planes of extension are arranged parallel to each other, and the carriers 3a, 3b are initially spaced far enough apart from each other that the second carrier 3b does not contact the microdroplets 2a, 2b located on the first carrier 3a. Then the carriers are moved toward each other, roughly normal to their planes of extension, until the second carrier 3b contacts the microdroplets 2a, 2b that are paired with each other and these two microdroplets come into contact with each other.

In FIG. 8 it can be seen that, after the microdroplets 2a, 2b are mixed together to form a new microdroplet 2, the carriers 3a, 3b are spaced apart from each other by means of a narrow intermediate space and that the microdroplets 2 are spaced apart from each other by means of the hydrophobic surface domain 6a. Thus, only the microdroplets 2a, 2b that are paired with each other are mixed together.

In the example of the embodiment shown in FIGS. 9 and 10, the arrangement of the surface domains 5a, 6a corresponds to that shown in FIG. 5. However, second hydrophilic surface domains 5b, which are spaced apart from each other by a second hydrophobic surface domain 6b, are provided on a second carrier. As in the embodiment shown in FIG. 5, the first hydrophilic surface domains 5a are coated with first and second microdroplets 2a, 2b. Third microdroplets 2c are applied to the second hydrophilic surface domains 5b.

FIG. 9 shows that the carriers 3a, 3b and their planes of extension are arranged parallel to each other such that the second hydrophilic surface domains 5b each overlap an area that is part of the first hydrophobic surface domain 6a and that is located between the first hydrophilic surface domains 5a. It can clearly be seen that the third microdroplet 2c in the top view looking down onto the planes of extension of the carriers 3a, 3b is located between a first microdroplet 5a that is associated with the third microdroplet 2c and a second microdroplet 5b. Here, the carriers 3a, 3b are spaced far enough apart so that the microdroplets 2a, 2b, 2c do not contact each other. The carriers are then moved toward each other normal to their planes of extension until the third microdroplets 2c contact the first and second microdroplets 2a, 2b and mix together with them.

In FIG. 10 it can be seen that, after the microdroplets 2a, 22b, 2c are mixed together to form a new microdroplet 2, the carriers 3a, 3b are spaced apart from each other by means of a narrow intermediate space and that the microdroplets 2 are spaced apart from each other by means of the hydrophobic surface domains 6a, 6b. Only three microdroplets 2a, 2b, 2c that are associated with each other are mixed together.

The first microdroplet 2a can contain hydrogen peroxide, the second microdroplet 2b can contain Luminol, and the third microdroplet can contain a serum that is to be tested in which ligands are marked with an enzymatic marker such as horseradish peroxidase (HRP).

1. A process for mixing microdroplets in which there are provided at least two carriers whose surfaces are each structured in such a way that at least one hydrophilic surface domain is delimited by at least one hydrophobic surface domain, in which a first microdroplet is disposed on a hydrophilic surface domain of a first carrier, and a second microdroplet is disposed on a hydrophilic surface domain of a second carrier, and in which the carriers, with their first and second hydrophilic surfaces facing each other, are positioned adjacent to each other and sufficiently close to each other by being moved toward each other in such a way that the microdroplets come into contact with each other.

2. The process of claim 1, wherein the surface of the first carrier is provided with a preferably matrix-like surface structure that has a plurality of hydrophilic surface domains that are separated from each other by at least one hydrophobic surface domain, the surface of a second carrier is provided with a surface structure that coincides with the surface structure of the first carrier, one microdroplet is applied to each of the individual hydrophilic surface domains, and the carriers, with their surface structures facing each other, are positioned adjacent to each other by being moved toward each other in such a way that the microdroplets of hydrophilic surface domains that correspond to each other come into contact with each other.

3. A process for mixing microdroplets in which at least two carriers are provided, in which the surface of a first carrier is structured in such a way that hydrophilic surface domains that are adjacent to each other and close to each other are separated from each other by at least one hydrophobic surface domain, in which the hydrophilic surface domains are each brought into contact with a microdroplet, and in which a second carrier is positioned relative to the hydrophilic surface domains in such a way that the microdroplets come into contact with the second carrier and with each other.

4. A process for mixing microdroplets, in which at least two carriers are provided, in which the surface of a first carrier is structured in such a way that first hydrophilic surface domains that are adjacent to each other and close to each other are separated from each other by at least one first hydrophobic surface domain, in which the surface of a second carrier is structured in such a way that at least one second hydrophilic surface domain is delimited by at least one second hydrophobic surface domain, in which the first hydrophilic surface domains (5a) and the second hydrophilic surface domain, of which there is a least one, are each brought into contact with a microdroplet and in which the carriers, with their first hydrophilic surface domains and the second hydrophilic surface domain, of which there is at least one, facing each other, are positioned adjacent to each other and sufficiently close to each other by being moved toward each other, that the second hydrophilic surface domain overlaps an area of the first hydrophilic surface domain that is located between the first hydrophilic surface domains, and the microdroplets, of which there are at least three, come into contact with each other.

5. The process of claim 1, wherein at least one of the carriers for applying the microdroplet(s) onto the hydrophilic surface domain(s) is immersed into a liquid and then preferably drawn out of the liquid at a speed in the range of 0.1 to 10 mm/seconds.

6. The process of claim 1, wherein a first microdroplet contains enzymes and a second microdroplet contains at least one DNA molecule, primer, and nucleoside triphosphate in a sufficient concentration for a polymerase chain reaction.

7. The process of claim 1, wherein a first microdroplet contains hydrogen peroxide, and a second microdroplet contains Luminol.

8. The process of claim 1, wherein at least one carrier is provided as a metal oxide substrate or semi-metal oxide substrate, and the substrate is coated with at least one polymer possessing a reactive group at the locations at which the hydrophilic surface domains are to be provided.

9. A device to mix microdroplets having at least two carriers whose surfaces are each structured in such a way that at least one hydrophilic surface domain is delimited by at least one hydrophobic surface domain, and having a positioning means, by which means the carriers, with their structured surfaces facing each other, may be positioned adjacent to each other and sufficiently close to each other that the microdroplets that can be applied to the hydrophilic surface domains come into contact with each other.

10. A device to mix microdroplets having at least two carriers in which the surface of the first carrier is structured in such a way that hydrophilic surface domains that are adjacent to each other and located close to each other are separated from each other by at least one hydrophobic surface domain, and having a positioning means, by which means the carriers may be positioned adjacent to each other and sufficiently close to each other that the microdroplets that can be applied to the hydrophilic surface domains of a first carrier come into contact with the second carrier and with each other.

11. A device for mixing microdroplets, having at least two carriers, in which the surface of a first carrier is structured in such a way that first hydrophilic surface domains that are adjacent to each other and close to each other are separated from each other by at least one first hydrophobic surface domains, in which the surface of a second carrier is structured in such a way that at least one second hydrophilic surface domain is delimited by at least one second hydrophobic surface domain, and having a positioning means, by which means the carriers, with their structured surfaces facing each other, can be positioned adjacent to each other and sufficiently close to one another that the second hydrophilic surface domain overlaps a first hydrophilic surface domain that is located between the first hydrophilic surface domains, and that microdroplets that can be applied to the first hydrophilic surface domains come into contact with microdroplets that can be applied to the second hydrophilic surface domains.

12. The device of claim 9, wherein said device has at least three of the carriers, and said carriers may be positioned adjacent to each other using the positioning means, either as desired or alternatively.

13. The device of one of claim 9, wherein at least one carrier has a metal oxide substrate or semi-metal oxide substrate that is coated on the hydrophilic surface domains with a polymer possessing at least one reactive group.

14. The device of claim 9, wherein the positioning means has centering elements that work together with each other on the carriers that are to be positioned adjacent to each other, in particular inclined centering surfaces.

15. The device of claim 9, wherein at least one carrier has a moisture and or conductivity sensor, preferably on a hydrophilic surface domain.

16. The device of claim 9, wherein at least one carrier has a cooling and/or heating element, in particular a Peltier element.

17. The process of one claim 3, wherein at least one of the carriers for applying the microdroplet(s) onto the hydrophilic surface domain(s) is immersed into a liquid and then preferably drawn out of the liquid at a speed in the range of 0.1 to 10 mm/seconds.

18. The process of claim 4, wherein a first microdroplet contains enzymes and a second microdroplet contains at least one DNA molecule, primer, and nucleoside triphosphate in a sufficient concentration for a polymerase chain reaction.

19. The device of claim 11, wherein at least one carrier has a metal oxide substrate or semi-metal oxide substrate that is coated on the hydrophilic surface domains with a polymer possessing at least one reactive group.

20. The device of claim 11, wherein the positioning means has centering elements that work together with each other on the carriers that are to be positioned adjacent to each other, in particular inclined centering surfaces.
